(19) United States
(12) Patent Application Publication
(10) Pub. No.: US 2025/0339070 A1
(43) Pub. Date: Nov. 6, 2025

(54) DEVICE AND METHOD FOR ACUTE STRESS ASSESSMENT

(71) Applicant: NANOMADE LAB, TOULOUSE (FR)

(72) Inventor: Adrien THIRION, TOULOUSE (FR)

(73) Assignee: NANOMADE LAB, TOULOUSE (FR)

(21) Appl. No.: 18/655,113

(22) Filed: May 3, 2024

Related U.S. Application Data
(63) Continuation-in-part of application No. 18/692,930, filed on Aug. 13, 2024.

(51) Int. Cl. A61B 5/16; A61B 5/00; A61B 5/18; G16H 50/30

(52) U.S. Cl. CPC A61B 5/165; A61B 5/18; A61B 5/4884; A61B 5/6891; A61B 5/7225; A61B 5/7267; A61B 5/746; G16H 50/30; A61B 2503/22; A61B 2562/0247

(57) ABSTRACT

A method for assessing a state of stress of an individual when the individual is contacting a receiving surface of a device comprising a pressure sensor comprising a plurality of elemental gauges, each elemental gauge having a gauge factor of at least 10, the method comprising acquiring and digitizing each signal delivered by each elemental gauges of the pressure sensor each signal comprising a pseudo-periodic part, exploring the signal and detecting patterns corresponding to heartbeats, measuring an Inter-Beat interval and recording in a heartbeat timestamped file each Inter-Beat Interval, from the heartbeat timestamped file computing at least 3 stress indices and categorizing the state of stress of the individual based on the at least three indices and a stress assessment program implementing a trained algorithm, among an increasing stress, a decreasing stress and a neutral state.

### CROSS-REFERENCE TO RELATED APPLICATIONS

The instant application is a Continuation In Part of U.S. application Ser. No. 18/692,930 filed on Mar. 18, 2024, which is a § 371 application of PCT/EP2022/064356 filed on May 26, 2022, which claims priority of French Patent Application No. 2111154 filed on Oct. 10, 2021, each of which is incorporated herein by reference in its entirety.

### BACKGROUND OF THE INVENTION

The invention is in the field of information and communication technology for calculating health and well being indices of an individual based on data automatically collected from this individual through non-obtrusive sensors.

The invention pertains to a method and a device for assessing a state of acute stress, and thus anxiety and discomfort, of an individual being in short duration contact with a receiving surface, like the seat or the backrest of a chair. A short duration stands for a duration in the range of minutes, typically in the 2 to 30 minutes range, as compared with wearables like wrist bands, smart watches or smart glasses that an individual may wear for a whole day long. Yet, the device and the method are not limited to such a short duration contact and may also be used for an assessment over a longer period of time, either continually or by intermittent contacts with the device.

Acute stress is a common type of stress, experienced by people a few times each day. In some instance, a situation of acute stress that lasts too long may lead to an uncomfortable anxiety. As a for instance, it may be the case for a passenger of an aircraft suffering flight anxiety.

In such an example, it could be useful for a flight attendance crew to be able to discreetly detect such a flight anxious passenger and helping him/her to have a more pleasant experience.

On the aircraft pilot side, a proper stress management is paramount in decision making and avoiding so-called tunneling effect in a situation assessment.

There are multiple other instances in everyday life where being able to assess the stress of an individual may help a person interacting with such an individual experiencing an acute stress situation, for example in medical teleconsulting or in a law enforcement context, to adapting its behavior accordingly.

The measurement of physiological parameters such as related to a heart rate, a respiratory rate, a blood pressure and their temporal changes, makes it possible, at least in laboratory conditions, to assess a state of stress of an individual and using this information to improve the well being of this individual.

To spread such technologies out of the laboratory, difficulty lays first in the collection of the physiological information.

Devices, frequently referred to as “wearables”, such as a wrist band or a chest belt, can measure such parameters relating to the physical-psychic state of the person wearing them.

In a laboratory environment, different types of sensors may be used to assess and monitor physiological parameters of an individual such as an electrocardiogram (ECG), an electroencephalogram (EEG), a respiratory or blood pressure monitoring.

However, these technical means are not suitable for the examples cited above, due to the discomfort they produce in daily activities and the feeling of surveillance they raise by their simple presence.

Furthermore, for the implementation of currently available technologies, parameters of interest are requiring, for their measurement, the sensors to be in contact with the skin of the individual and are usually responsive to environmental parameters such as temperature or humidity.

Therefore, the techniques that may reliably be implemented in laboratory conditions, are delicate to implement in a stand-alone sensor, and more particularly if the latter is not a wearable.

On another hand, sensors that are not in direct contact with the skin of the individual and therefore more remote from the phenomenon to be sensed are less responsive to small variations of the physiological parameter and more prone to noise, i.e. the signal-to-noise ratio is less favorable for fine tuned measurement.

Furthermore, the assessment of a stress level may require historical data about the individual, such as biographical and physical information such as age, weight or medical conditions, but also prior records about the measured parameters or signals, like ECG or blood pressure. As a matter of fact, signal features that may depict a high acute stress level for a given individual may correspond to a mild or even low state of stress for another individual and vice versa.

Basically, an acute stress state may be detected and assessed by comparison with a steady state assumed as stress free and, today, this is performed through a specific and well-designed protocol in controlled conditions.

Those issues may be experienced individually or in combination at different relative levels depending on the sensing technology, the environment of the test, the degree of automation of the assessment and the allowed or foreseen duration of the data analysis to reach a conclusion/assessment.

### SUMMARY OF THE INVENTION

The disclosed device and method aim at solving these shortcomings by enabling an assessment of a state of stress of an individual by a short duration contact with a non-obtrusive equipment such as a seat or a bed.

To this end, a highly sensitive sensory, a dedicated signal processing and a feature interpretation algorithm based on machine learning, implemented through a computerized system either on site, remote or distributed between local and remote, may be implemented alone or in combination.

Therefore, the invention pertains to a method for assessing a state of stress of an individual when the individual is contacting a receiving surface of a device comprising a pressure sensor comprising a plurality of elemental gauges, an elemental gauge having a gauge factor of at least 10, the pressure sensor being responsive to a pressure on the receiving surface and delivering a signal to a computer comprising and acquisition and digitization board a non-transitory memory and a computer program configured for processing the signal delivered by the pressure sensor, the method comprising steps of:
acquiring and digitizing a signal delivered by an elemental gauge of the pressure sensor the signal comprising a continuous part and a pseudo-periodic part;
in a preprocessing step, extracting the pseudo-periodic part of the signal;
filtering by a bandpass filter with cut-off frequencies of 0.5 Hz and 20 Hz the pseudo-periodic part of the signal obtained in the preprocessing step to obtain a filtered signal;
selecting the filtered signal having a best signal-to-noise ratio among the plurality of elemental gauges;
exploring the selected filtered signal by a sliding window and detecting, in the sliding a window, two patterns corresponding to heart beats, measuring an Inter-Beat interval of time between these two patterns; and
recording in a heartbeat timestamped file each Inter-Beat Interval (IBI) thus obtained and a corresponding date of measurement.

The gauge factor is the ratio between the variation of an electrical property measured at the terminals of the gauge, generally a resistance, and the variation of deformation of this gauge.

The invention may be implemented according to the embodiments and variants exposed hereafter which are to be considered individually or according to any technically operative combination.

According to an embodiment, the elemental gauge comprises an assembly of electrically conductive nanoparticles in an electrically insulating ligand and two electrically conductive electrodes having a comb shape and deposited on the assembly of electrically conductive nanoparticles in a nested interdigitated configuration.

This configuration allows to reach a high gauge factor for the elemental gauge.

Advantageously, the gauge factor of the elemental gauge is at least 80.

According to a specific embodiment, the device is in a form of a chair comprising a backrest and a seat and comprising a backrest pressure sensor in the backrest and a seat pressure sensor in the seat.

According to another specific embodiment, the device is in a form of a mattress comprising a sleeping surface and wherein the sleeping surface is the receiving surface.

According to an embodiment, the method further comprises the steps of:
from the heartbeat timestamped file computing at least three indices over an assessment time window comprising an average value of the IBI over the assessment time window, a standard deviation of the IBI over the assessment time window and a Baevsky's stress Index from the IBI over the assessment time window;
categorizing a state of stress of the individual based on the computed indices and a stress assessment program implementing a trained algorithm, among an increasing stress, a decreasing stress and a neutral state; and
the assessment time window is comprised between 25 seconds and 250 seconds.

Advantageously, the trained algorithm is an Extreme Gradient Boosting algorithm.

According to a specific embodiment, a training set for training the trained algorithm is built by:
submitting a selection of subjects to a stress test comprising at least a neutral phase, an increasing stress phase and a decreasing stress phase
continuously recording an IBI of the selection of subjects during their performance of the stress test; and
a duration of the stress test comprised between 10 minutes and 30 minutes and a duration of each phase is at least 4 minutes.

Advantageously, a continuous recording of an IBI of a subject is spilt in windows of a duration comprised between 25 seconds and 250 seconds, a beginning of each window being separated from a beginning of a previous window by 5 seconds, the at least three indices being computed for each window.

The increasing stress phase may be obtained by submitting the selection of subjects to a stressor exercise.

The stressor exercise may comprise a modified Stroop test wherein the selection of subjects is given a limiting time to reply to each Stroop test exercise.

According to a specific embodiment, the device is in a form of an aircraft seat and the individual is seating on the seat, the method comprising the step of:

If the state of stress of the individual is categorized as an increasing state of stress for more than an uninterrupted limited time set, triggering an alarm.

Advantageously, the recording of the heartbeat timestamped file is stopped if an intensity of the pseudo—periodic part of the signal of the plurality of elemental gauges remains under a minimum threshold for more than a given unoccupancy time.

According to an embodiment the device is in a form of an aircraft seat and the individual is a pilot seating on the aircraft seat, the heartbeat timestamped file is recorded in a flight data recorder.

### BRIEF DESCRIPTION OF THE DRAWINGS

The device and method are implemented according to the preferred embodiments, in no way limiting, exposed hereafter with reference to FIG. 1 to FIG. 11 in which:

FIG. 1 shows in a perspective view an exemplary embodiment of the device implemented in a chair;

FIG. 2 shows, in an exploded perspective view an exemplary embodiment of a pressure sensor implemented in the device;

FIG. 3 represent schematically s, according to an exploded perspective view, an exemplary embodiment of an elemental gauge of a sensor of the device;

FIG. 4 is a flowchart of the method;

FIG. 5A shows an example of a digitized raw signal received from all the elemental gauges of a sensor of the device;

FIG. 5B shows an example of the digitized raw signal on an elemental gauge and illustrates the processing performed on this signal;

FIG. 6 shows an example of a signal processed for the determination of a respiratory rate;

FIG. 7 illustrates a processing performed on the signal to obtain a BCG, in order to eliminate the influence of micromovements;

FIG. 8 shows a qualitative evolution of a state of acute stress level of a subject during a training for a machine learning of a stress detection algorithm;

FIG. 9 Illustrates the processing performed in a BCG signal exploration window in order to determine the distances between peaks;

FIG. 10 illustrates a signal processing from an elemental gauge for the analysis of the posture of the individual in contact with the device of the invention;

FIG. 11 shows a principle view a use case of the method and the device for assessing a state of stress of a flight passenger.

### DETAILED DESCRIPTION OF THE INVENTION

### Description of the Sensor

The implemented pressure sensor exhibits a combination of:
high sensitivity;
extended deformation measurement range,
mechanical flexibility allowing it to be integrated, without discomfort for a user, into any piece of furniture comprising a receiving surface, with or without padding.

These characteristics enable the sensor to detect the presence of an individual in contact with the receiving surface of a piece of furniture thus equipped, even to detect the posture of this individual by the distribution of pressure on the receiving surface, but also to measure the pressure variations generated on this receiving surface by the heartbeat, blood circulation and breathing of this individual regardless of the weight of the latter.

Thus, according to nonlimiting examples, the receiving surface is:
a backrest or a seat of a chair or a bench with or without padding;
a sleeping surface of a bed or a berth;
an area delimited on a floor, covered or not with a carpet or a coating.

Consequently, the device is applicable in any furniture, at home or onboard, in particular in a driver or passenger seat in the field of transportation, work equipment, sport and leisure or devices intended for people with reduced mobility, in a mattress, in particular intended for a hospital bed or for a home hospitalization.

In a specific embodiment the sensor is embedded in a passenger seat of an aircraft.

The sensitivity of the pressure sensor and associated method make it possible to acquire a signal corresponding to a balistocardiogram (BCG) of a person coming into contact with a receiving surface thus functionalized.

Compared to an ECG or electrocardiogram, BCG is much less responsive to environmental parameters such as humidity and does not require direct contact with the individual's skin.

On the other hand, the measurable signal is more responsive to phenomena such as noises, vibrations or changes in the posture of the person in contact with the receiving surface, and in general, the signal-to-noise ratio of the relevant information is less favorable than for an ECG. This drawback is solved by the signal processing method.

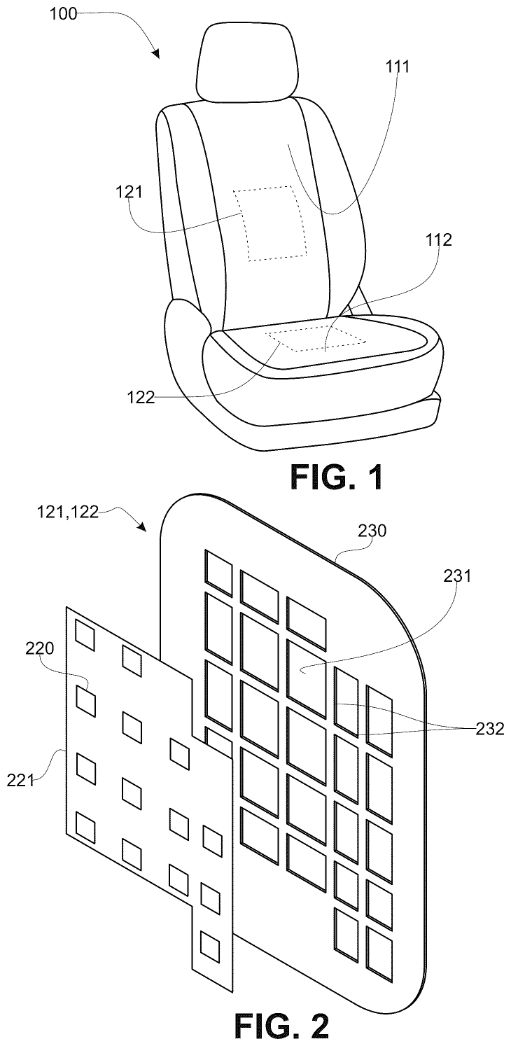

FIG. 1, according to an exemplary embodiment, a piece of furniture such as a chair or a seat (100) comprises one or more receiving surfaces (111, 112) aimed to coming into contact with a body part of a user when the latter uses the piece furniture.

According to this exemplary embodiment, the backrest and seat of the chair are receiving surfaces with padding, into which pressure sensors (121, 122) are inserted.

FIG. 2, according to an exemplary embodiment, the pressure sensor (121, 122) comprises one or more elemental strain gauges (220) attached, for example by bonding, to the back of a thin polycarbonate plate (230), for example with a thickness of less than or equal to 0.5 mm, acting as a test body. The elemental gauges (220) are deposited on a thin insulating substrate (221) for example using a capillary/convective deposition technique or by soft lithography.

According to an exemplary embodiment, the elemental gauges (220) are arranged on the back of the polycarbonate plate (230) so as to be protected by the polycarbonate, for example at the intersections of the strips (232) delimiting the cuts (231).

According to a particular example, the sensor (121, 122) comprises 12 elemental gauges.

The sensor further comprises a circuit and electrical connections (not shown) suitable for acquiring the information delivered by the elemental gauge(s) (220), the circuit and the connections being, at least in part, deposited on the substrate (221), also by soft lithography or photolithography techniques, according to exemplary implementations.

The polycarbonate plate (230) thus equipped is, for example, integrated into the padding of a seat or a backrest of a chair, the face opposite the one comprising the elemental gauge(s) (220) being turned towards the individual likely to use the chair.

The low thickness of the sensor and its shape, including for example cut-outs (231) improving its flexibility, does not cause any discomfort and does not impair the comfort of use of that chair compared to an ordinary chair.

According to another embodiment (not shown), corresponding to the case where the piece of furniture comprises a rigid receiving surface, for example the unpadded backrest of a chair, the elemental gauges are directly attached to the rigid surface, for example on the back of the backrest, protected from the environment by a protective coating.

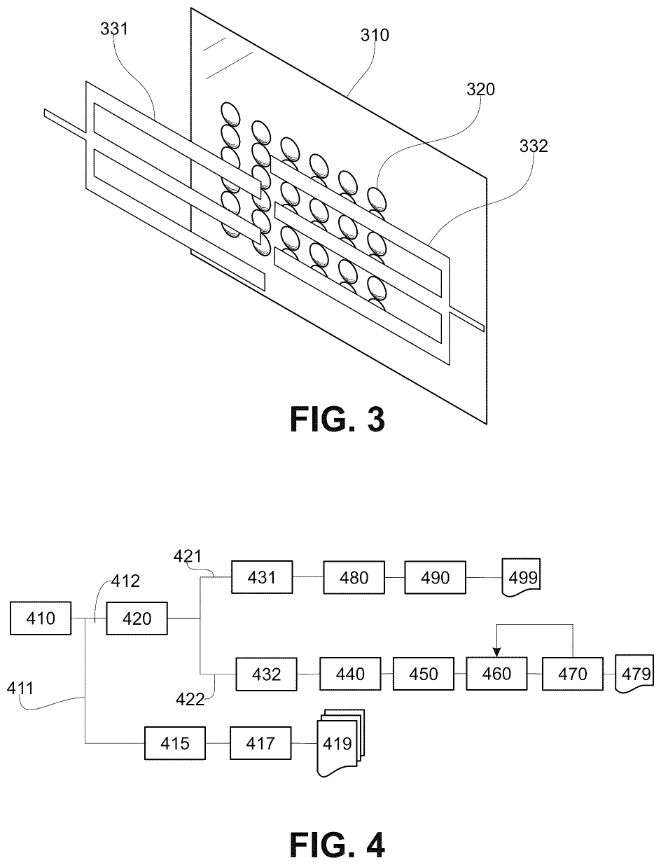

FIG. 3, according to an exemplary embodiment, an elemental gauge (220) comprises a substrate (310) on which is deposited an assembly of electrically conductive or semiconductive nanoparticles (320) in an electrically insulating ligand capable of binding to the surface of the nanoparticles.

By way of nonlimiting examples, the nanoparticles (320) consist of zinc oxide (ZnO) or indium tin oxide (In₂O₃—SnO₂), or ITO. For example, the substrate (310) consists of ethylene poly (terephthalate) (PET), the ligand is for example based on phosphonic acid.

The nanoparticles are attached to the substrate by a graft, by means of a chemical coupler, for example a silane.

These deposition techniques, both assemblies of nanoparticles and electrodes or electrical circuit elements, are known from the prior art, in particular from documents U.S. Pat. No. 9,436,215 B2 and U.S. Pat. No. 10,318,143 B2 which are hereby incorporated by reference.

Two comb shaped electrically conductive electrodes (331, 332), for example made of ITO, are deposited on the assembly of nanoparticles (320) in a nested configuration, called interdigitated, that is to say the teeth of one of the comb shaped electrodes are interposed between the teeth of the other comb shaped electrode.

Thus, according to this exemplary embodiment, each tooth of a comb juxtaposed with a tooth of the other comb defines between the teeth a micro strain gauge which is the place of electrical conduction by tunneling between the nanoparticles of the assembly located between the electrodes delimiting the micro-gauge. The conduction varies according to the distance between the nanoparticles of the assembly, which distance is a function of the pressure applied to the assembly of nanoparticles, or more generally to the deformation to which the gauge (220) is subjected.

Thus, the conductivity or resistance of the gauge (220) varies with this deformation.

A passivation layer (not shown) consisting, for example, of a polyimide, is placed on this stack in order to protect it from the environment, in particular from moisture.

The gauge factor defines the ratio of the relative change in the resistance of elemental gauge ΔR/R0 according to the relative deformation of the gauge. This gauge factor easily reaches 80 or more over a deformation range of ±1%, the resistance R0 of such a gauge comprising nanoparticles of ITO in a ligand based on phosphonic acid, exceeds 2000 ohms in the absence of deformation.

Such an elemental gauge (220) exhibits a high sensitivity, and the sensitivity of the sensor is further improved by the combination of several of these elemental gauges with a suitable test body.

### Signal Processing

The high sensitivity of the sensor makes it possible to detect the presence of an individual on the piece of furniture, the movements or micromovements of this individual, his posture and his changes in posture, his heart rate by BCG and the variation of this heart rate, as well as the respiratory rate of this individual and its variation.

The person skilled in the art understands that this various information is contained in a signal originating from the sensor and that it may be extracted from this signal by an adapted processing.

This processing is carried out by computer means including, according to a known general configuration, means of acquisition and digitization of signals, means of calculation and means of non-transient memory, all being controlled by a computer program.

Such IT means include a clock so that any acquisition and storage of data, raw or processed, can be associated with a date and that this date can be used for any processing, in particular those aimed at determining an evolution.

FIG. 4, according to a first digitizing step (410) the signals issued by the elemental gauges of the sensor are digitized according to methods known to the prior art in order to apply adapted digital processing to the digitized signals.

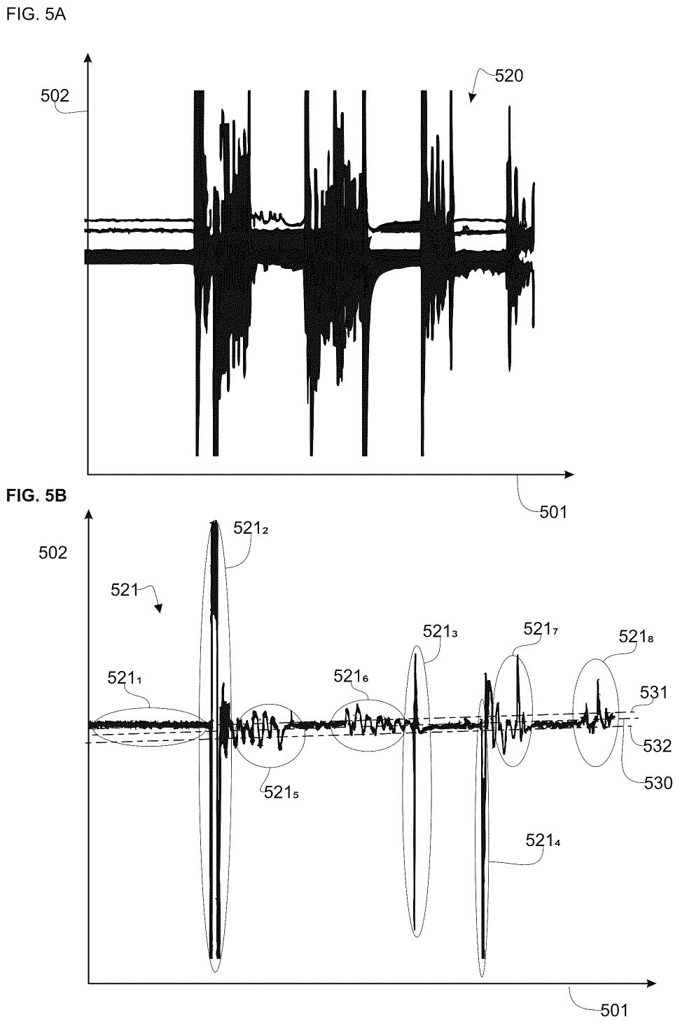

FIG. 5A shows an example of the change in the amplitude (502) of raw signals (520) as a function of time (501), these raw signals (520) may represent pressures or accelerations.

Thus, in a graph showing a sensor emitted raw signal (520) amplitude (502), for example in volts, as a function of time (501), wherein an elemental gauge emits such a signal comprising a combination of different aimed information (movements, posture, heart rate, respiratory rate), such different information produces events that differ in amplitude, frequency, and reproducibility.

Thus, for example, heartbeats as well as breathing correspond to pseudo-periodic events, while the movements or micromovements of the individual are more stochastic, the movements producing variations of higher amplitude and micromovements producing information whose amplitude is between that of the heartbeats and that of breathing.

For example, FIG. 5B, the observation of gauge signal (521) corresponding to a single elemental gauge makes it possible, under experimental conditions, to identify specific events. A first portion (521₁) of this gauge signal corresponds to the absence of contact of the individual with the receiving surface.

The next portion (521₂) corresponds to the detection of a movement, for example, when the individual sits in the chair of FIG. 1.

Other events (521₃, 521₄) also correspond to movements of the individual.

The pseudo-periodic parts (521₅, 521₆, 521₇, 521₈) of the signals correspond to breathing signals, while the signal portions between these breathing phases correspond to a situation where the subject is in contact with the receiving surface but blocks his breathing.

The signals corresponding to heart beats and possible micromovements are present but are not visible graphically at the scale of FIG. 5B.

The time drift of the signal of the corresponding gauge is estimated by a linear regression producing a regression line (530) over a defined time window, typically greater than 3 seconds and less than 60 seconds and in the order of 20 seconds.

Additionally, an upper minimum threshold (531) and a lower minimum threshold (532) may be defined on the upper side and the lower side of the regression line (530).

These minimum thresholds (531, 532) may be defined for a specific environment depending on the noise and vibration level of this environment. If the intensity of the pseudo periodic signal remains inside this upper and lower boundary for a significant period of time, this may be an indication that the individual as left contact with the pressure sensor or that an item exercising a pressure on the pressure sensor is a dead weight, like a bag set in a seat, and not a living individual.

FIG. 10, similar to FIG. 5B, corresponds to an exemplary observation of the amplitude (1002) of a signal (1020) corresponding to an elemental gauge as a function of time (1001), from which the time drift has been deduced.

By comparing the average signal level (1021₁) before the individual comes into contact with the piece of furniture to the average signal level (1021₂) after the individual comes into contact with the piece of furniture, the difference (1030₁) gives the corresponding pressure exerted on the elemental gauge from which the signal is originated.

Then, during a subsequent movement of the individual, to see if this pressure (1030₂) changes.

By performing this operation on a plurality of elemental gauges of the sensor, and by successive time windows, the distribution of these measurements provides information on the posture of the individual, that is to say on the distribution of the pressure that he exerts on the piece of furniture (chair, bed . . . ) and consequently, the evolution of this information over time, provides information on the changes, or stays, of the individual posture.

Returning to FIG. 4, the signal may be processed according to two branches.

According to an optional processing branch (411) aimed at extracting information relating to the posture of the individual on the piece of furniture, during a resetting step (415) the drift of the signal is estimated over a defined time range, typically in the 20 seconds order of magnitudes, for example by a linear regression over the raw signal, and the corresponding straight line is deduced from the signal over the duration of the defined time range.

During an assessment step (417), the average pressure, the average level differences of this corrected signal in successive observation time windows are compared, these differences are stored in an actimetry timestamped file (419) for each elemental gauge.

The data in the actimetry timestamped file (419) may be analyzed according to a temporality specific to the application of the device, to derive information on the individual's posture and its evolution, which reflects the individual activity which may be used as part of a state of stress determination.

According to another branch (412) the processing aims at extracting from the signal information relating to a heart rate and to a respiratory rate, this branch (412) comprises a step of preprocessing (420) the raw signals issued by the elemental gauges of the sensor.

The preprocessing carried out during this preprocessing step (420) aims to separate the influence on the signals of the part corresponding to pseudo-periodic phenomena such as breathing and heart rate, from the part related to movements of the individual in contact with the receiving surface. According to this example, this step may be carried out by an analysis of a signal amplitude threshold.

Thus, to isolate the pseudo-periodic part of the signals, the signals of the plurality of elemental gauges are set to 0 each time a peak in the digitized raw signal crosses a defined threshold and for a duration around this peak, for example for a duration ranging from 1 second before the phenomenon (peak) to 3 seconds after this phenomenon.

For example, a peak in the signal is assigned to a movement if the full amplitude of the signal (max-min) over a range of 1 second exceeds a threshold for signals issued from at least two elemental gauges and preferentially from at least 3 elemental gauges.

After this preprocessing step (420) the majority of the peaks attributable to movements are eliminated from the signal. The person skilled in the art understands that the signal corresponding to these movements can be extracted from the raw signal by proceeding in a similar way.

This preprocessing method makes it possible to eliminate signals that are irrelevant to the intended objective, in this case, signals corresponding to movements, when the intended analysis is focused on BCG or respiratory rate, without shifting the phase of the signal according to the frequency.

Starting from this preprocessing step, the signal may follow two processing branches (421, 422) a breath analyzing branch (421) corresponding to a processing aiming at a respiratory rate assessment and a heartbeat analyzing branch (422) for a processing aiming at a BCG assessment.

Thus, on the breath analyzing branch (421) corresponding to the processing of a respiratory rate, the preprocessed signal is the subject of a smoothing step (431), for example via a Savitzky-Golay algorithm with a 3^{rd} degree polynomial and a smoothing window of 1 second.

On the heartbeat analyzing branch (422) corresponding to the processing of a BCG, the preprocessed signal is subjected to a filtering step (432) in the form of a bandpass filter with exemplary cut-off frequencies of 0.5 Hz and 20 Hz.

According to exemplary embodiments, this filtering step (432) on the heartbeat analyzing branch (422) implements a Butterworth or Savitzky-Golay type filtering without these examples being limiting.

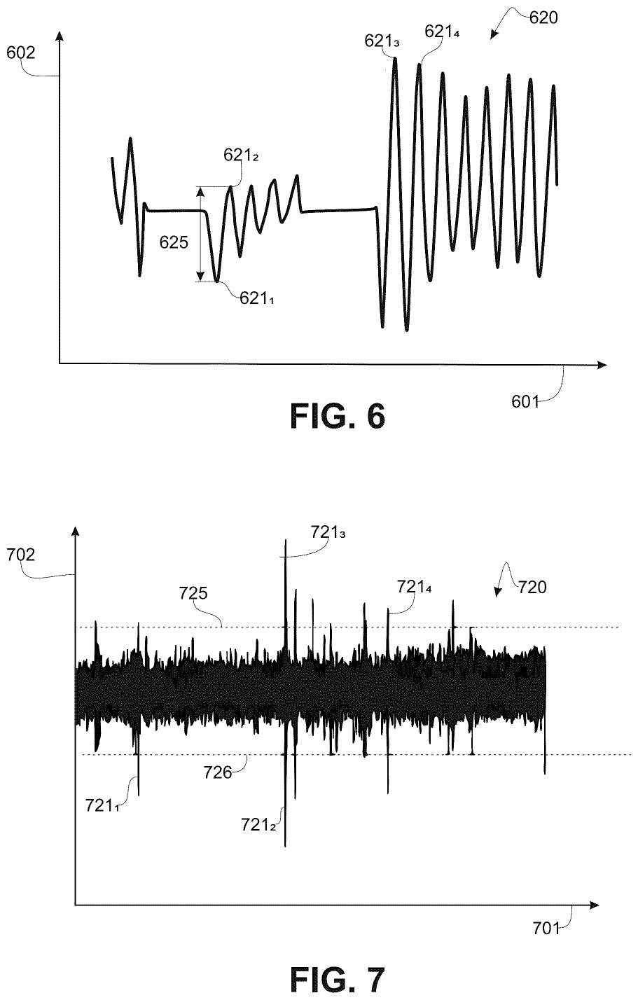

FIG. 6, according to an example, the signal (620) preprocessed according to the smoothing step (431) comprises peaks (6211, 6212, 6213, 6214) appearing as a function of time (601) in the signal amplitude (602), corresponding to respiratory events (inhaling or exhaling).

For example, a respiratory event corresponds to two successive peaks (6211, 6222), oriented in opposite directions, spanning over an amplitude (625) of magnitude defined by experience.

As a nonlimiting example, the amplitude (625) for the selection of significant peaks is defined relative to the signal.

Thus, the amplitude (625) for peak selection is equal to ⅕^{th} of the maximum amplitude of the signal over a given measurement range.

The duration of this measurement range is chosen inside a time frame of 2T_max, defined below, i.e. of the order of 3 seconds and a maximum of 60 seconds, preferably around 20 seconds.

Thus, going back to FIG. 4, a peak detection step (480) for detecting the breathing peaks, for example using the method described above, follows the smoothing step (431) of the signal.

According to a rate assessing step (490) for estimating the individual's respiratory rate, the respiratory rate is determined from the results of the peak detection step (480) on successive time windows and the corresponding results are recorded in a breath timestamped file (499).

Thus, by way of example, FIG. 6, to determine a respiratory rate, the time distances between two successive valid peaks (6212, 6213, 6214) are determined over a defined time range, the respiratory rate is determined by taking the inverse of the median of these distances over the given time range.

FIG. 4, the values relating to the individual's respiratory rate, stored in the breath timestamped file (499) may be analyzed according to a specific method as part of a state of stress determination.

A heartbeat analyzing branch (422) of the method corresponds to the processing of a BCG.

The mechanical phenomena corresponding to a BCG and captured by the elemental gauges produce a lower signal-to-noise ratio than those relating to breathing and therefore require more sophisticated processing in order to isolate cardiac phenomena and assessing the heart rate and its evolution.

The signal thus preprocessed is analyzed to determine the BCG only if:
it is free of movement phenomena for a predefined duration comprised between 2T_max (3 seconds) and 60 seconds, preferably 20 seconds, and
the presence of the individual on the receiving surface is confirmed.

As the movements are eliminated and the signal filtered, the signal is still likely to include information corresponding to the micromovements of the individual.

Thus, during a micromovement identification step (440), the preprocessed signal is filtered and analyzed.

FIG. 7, in an exemplary time (701)—amplitude (702) diagram, showing the preprocessed and filtered signals (720) from the elemental gauges, the peaks (721₁, 721₂, 721₃, 721₄) exceeding a given threshold (725, 726) are attributed to micromovement of the individual in contact with the receiving surface of the device.

The given thresholds (725, 726) may be defined by prior calibration tests depending on the application and may also take into account the environment of the device such as the presence of vibrations, in particular when the device is intended to be installed in a transportation mean.

In a first embodiment, once the thresholds are defined for a given application over an average population, they are set once and for all for such an application, for example for assessing a state of stress of an aircraft passenger on a passenger seat.

In another embodiment such as for teleconsulting such thresholds may be calibrated through a calibration protocol for a given individual in order to fine tune the signal processing for this individual and may also be checked and updated for time to time.

The thresholds values are stored in the non-transient memory of the IT system.

Thus, to isolate the pseudo-periodic part of the signals, the signals of all the elemental gauges are set to 0 each time a peak (721₁, 721₂, 721₃, 721₄) in the preprocessed and filtered signal (720) crosses a defined threshold and for the duration of this peak, as well as for a defined time range around such a peak.

Through this processing, the influence of micromovements is eliminated from the signal intended for a BCG analysis.

The BCG is responsive to the posture of the person on the receiving surface so that, according to this posture, some elemental gauges of the sensor are more responsive than others to the heartbeat of the individual.

To this end, during a selection step (450), a signal issued by an elemental gauge exhibiting the best signal-to-noise ratio is selected.

The BCG analysis comprises calculating the distance between the signal peaks that correspond to blood expulsion from the heart ventricles also called IBI (Inter-Beat Interval) and the variation of IBI over time is commonly called HRV (Heart Rate Variability).

Although these events are graphically visible, and despite prior signal processing as described above, automatic processing remains subject to parasitic phenomena given the conditions of signal acquisition and the environment.

According to an exploration step (460), a section corresponding to a time range of the analyzed signal, i.e. comprised between 3 seconds (2T_max) and 60 seconds, preferably of a duration of 20 seconds, is analyzed, according to a sliding window. This exploration aims at detecting in such a window the existence of 2 patterns corresponding to heartbeats and the distance separating these patterns.

To this end, a signal exploration variable is defined by times T_min and T_max. As an example, T_min is taken equal to 0.6 s (i.e. 100 bpm) and T_max is taken equal to 1.5 s (i.e. 40 bpm)

A sliding window of width 2T_max is analyzed around the signal exploration point v.

In principle, the treatment aims to:
determining if there are at least two peaks corresponding to heartbeats timely separated by at least T_min and at most T_max in the window;
determining the time distance between these peaks.

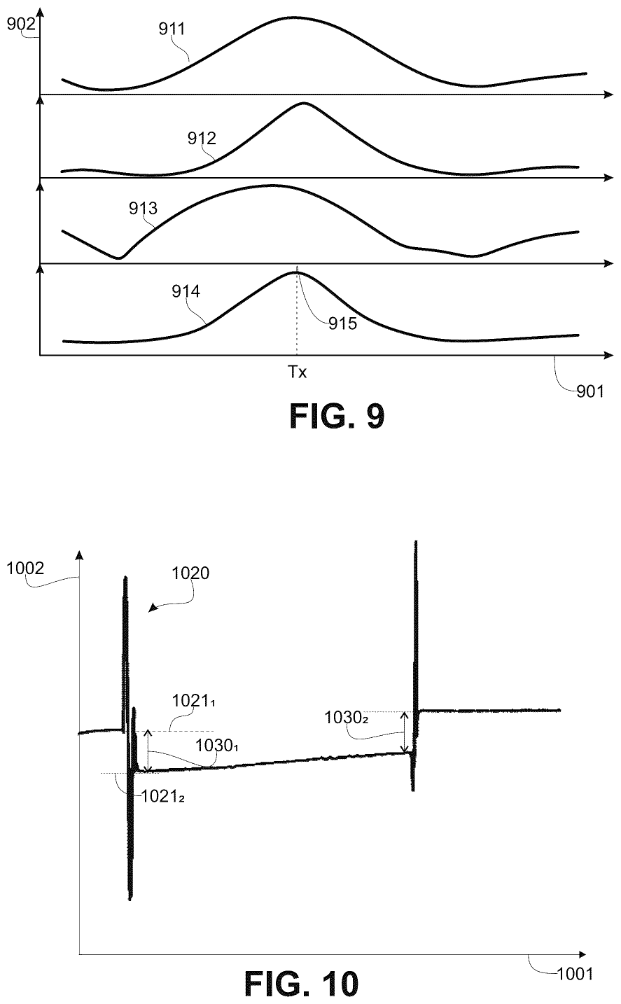

FIG. 9, according to an exemplary implementation, the processing consists in calculating on the window 2T_max, 3 functions having the signal as input:
an autocorrelation function (911);
a modified mean magnitude difference function (AMDF—912)
a maximum amplitude pair function (MAP—913)

These functions define a probability (902) of two peaks in the signal according to their time distance (901), by multiplying the probabilities (914) and taking a maximum probability (915) of the distance Tx separating two peaks.

During a verification step (470), the signal window analyzed at point v during the exploration step (460) is analyzed between v−Tx and v+Tx so as to locate the maximum amplitude peak in this range. The peak Px is identified for example by its time abscissa in the analyzed time range, preferably of a duration of the order of 20 seconds, and this information is recorded in a heartbeat timestamped file (479) where the Inter-Beat Interval is recorded with a corresponding date.

If there is already a peak with this same identification in the file, then it means that the pattern has already been identified during the analysis of a previous window. In this case, the determined Tx value is saved in a file. If a new Px peak is detected, then this is a new pattern. In this case, a value Tx corresponding to the median of the values Tx corresponding to the previous exploration windows is associated with the previous peak Px, and this median value of Tx is recorded in the heartbeat timestamped file (479).

If, however, such a peak is not detected, the Tx value determined is recorded in the heartbeat timestamped file (479). Tx is equivalent to IBI.

Therefore, at the end of this signal processing branch a heartbeat timestamped file (479) is obtained giving the IBI according to time through a corresponding date for each record.

Depending on the embodiment and the application of the system, each record may be appended to the heartbeat time stamped filed thus giving the IBI values over a potentially infinite time, which may make sense if these recordings correspond to a single individual, or the heartbeat timestamped file (479) may be limited to a certain duration ranging from minutes to hours e.g. for the duration of the flight.

Therefore, the signal processing method delivers at least one among a heartbeat timestamped file (479), a breath timestamped file (499) and an actimetry timestamped file (419).

Those files (479, 499, 419) may be sent in real time to a stress analyzing module or stored and processed offline by such stress analyzing module.

According to an embodiment the stress analyzing module uses an algorithm issued form machine learning.

### Machine Learning Scheme

As mentioned in the background art section, although a state of stress may be assessed based on a cardiac feature, a breathing feature and an actimetry feature taken alone or in combination, such assessments are usually requiring a reference specific to the individual that is the subject of the invention.

Getting such a reference is difficult, if not impossible. in some of the foreseen applications such as assessing the state of stress of a flight passenger.

To solve this issue the stress analyzing module assesses the stress of an individual based on an evolution i.e. increasing decreasing or steady state of stress.

A training set is built by subjecting a population of individuals to an activity aiming at generating stress from a quiet situation and to return to such a quiet situation. Therefore, the measurement cycle applied to an individual with the aim of generating a training set for the machine learning system may comprise at least a quiet or neutral phase, and increasing stress phase and a decreasing stress phase The entire cycle may last from 10 to 30 minutes preferably aroid 15 minutes. Meanwhile at least one stress indicative parameter is measured using appropriate means.

For instance, the IBI of the individual may be measured during the cycle, from which various indices may be calculated and used for machine learning.

For generating the training set, the IBI may be measured according to any known methods and does not require using the sensor and signal processing described above, instead an ECG using laboratory style means may be used.

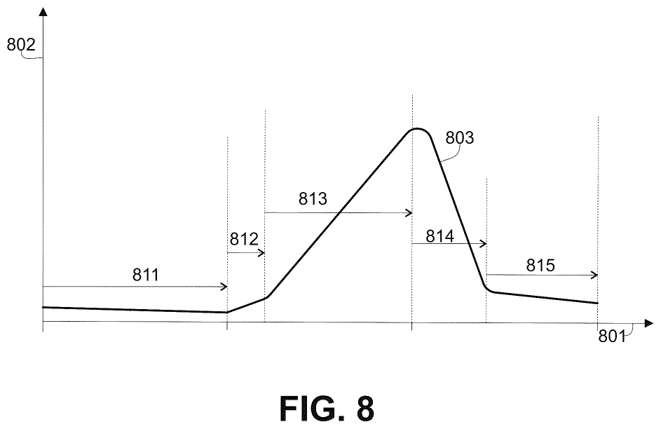

FIG. 8 shows according to time (801) an exemplary evolution of a stress level (803) in an arbitrary scale (802) of a subject subjected to a 15 minute cycle, performed according to an exemplary embodiment as follow: during a 5 minutes rest phase (811) the subject is only requested to stay calm without speaking or moving (e.g., reading a book). After the rest phase, a 1 minute instruction phase (812) follows, corresponding to the instructions for performing a stressor exercise. The instructions are not stated beforehand so as to avoid stressing the test subject during the rest phase. The stressor exercise (813) is performed for 4 minutes, at the end of the stressor exercise the subject is left to relax for 2 min during a relaxing phase (814), then a neutral phase (815) following the relax phase is recorded for 3 min.

A BCG or an ECG or at least an IBI are continuously recorded during the whole 15 minutes cycle.

A camera is set in front of the subject and displays his/her face which may also be recorded.

The subject is requested not to move nor speak during the test.

People behind the subject may comment on the subject's performance.

In an exemplary embodiment the stressor exercise consists in a modified Stroop test. A Stroop test itself is not designed to intentionally generate stress. Instead, it serves as a cognitive assessment tool. However, the Stroop test may be modified by giving the subject a limited time to provide an answer and instructing the subject to try to reach the highest score as possible or at least a minimum score.

For instance, the Stroop test may consist in a list of words printed in color, each word designating a color like red, orange, blue, green . . . but being printed in a different color than the designated one. For instance, the word “orange” may be printed in green. The subject is requested to name the color in which the word is printed. In the latter the subject should say “green” in less than a maximum allocated time, in order to score 1 point. If the subject announces “orange” or any other color than “green” he/she does not score and the same if he/she gives the right answer but after the allocated time.

The inventor found that such a modified Stroop test is reliable in producing an increasing acute stress level during the stressor exercise phase.

Therefore, for each individual carrying a test cycle, a continuous recording of one, e.g. IBI, or more parameters of interest are recording for the whole 15 minutes.

The recording is divided in successive sequences of assessment time windows, comprised between 10 seconds and 250 seconds and preferably of 25 seconds duration each window starting by a pitch duration, of e.g. 5 seconds, from the previous window beginning.

For instance, the first assessment time window starts a time 0 and ends at time 25 seconds, the second window starts at time 5 seconds and ends at time 30 seconds, the third window starts at time 10 seconds and ends at time 35 seconds and so on. Therefore, moving forward each recording provides 176 windows.

The recording may also be split in windows backward with a first backward window starting at time 900 seconds and ending at time 875 seconds, the second backward window starting at time 895 seconds and ending at time 870 seconds and so on, which gives an additional 176 windows per recording.

### Features Calculation

The following stress indices examples are given in the case of the heartbeat timestamped file processing. The person skilled in the art understands that the same or similar induces may be derived from the breathing and the actimetry recordings.

Taking the example of IBI the following exemplary stress indices may be computed for each window comprising N values of IBI:
average IBI value
standard deviation of IBI values
pNN50: the percentage of successive IBI pairs that differ for more than 50 ms
Root Mean square difference between successive IBI values
Standard deviation of successive IBI differences
Baevsky's stress Index or BSI with

$$BSI = \frac{100 . p A {M}_{0}}{2 . {M}_{0.} MxDMn}$$

where M₀ is a median of IBIs in the window, pAM₀ is obtained as an height of the normalized IBI histogram with a bin width of 50 m and MxDMn is a difference between the longest and the shortest IBI values.
HRV triangular index HTI with

$$HTI = \frac{N}{A {M}_{0}}$$

The recordings may also be analyzed according to frequency-based features, thus providing additional stress indices.

As a nonlimiting example two frequency bands may be considered namely a low frequency band (LF) for frequencies comprised between 0.04 Hz and 0.15 Hz and a higher frequency band (HF) comprised between 0.15 Hz and 0.4 Hz.

Examples of stress indices based on frequencies comprise:

An absolute power of combined LF and HF

$$Power = {\sum }_{f = 0.04 . N / {F}_{S}}^{f = 0.4 . N / {F}_{S}} {F}_{IBI} [ f ]$$

A relative power in LF:

$$L {F}_{P o w e r} = \frac{{\sum }_{f = 0.04 . N / {F}_{S}}^{f = 0.15 . N / {F}_{S}} {F}_{I B I} [ f ]}{Powe r}$$

Relative power in HF:

$$\begin{matrix}H {F}_{P o w e r} = 1 - L {F}_{P o w e r} \\ A \frac{L {F}_{P o w e r}}{H {F}_{P o w e r}} ratio\end{matrix}$$

Where IBI being defined as an array of time intervals between 2 heartbeat patterns, N is the number of values inside IBI, and F_{IBI} is a spectrum of IBI converted to equidistantly sampled series by an interpolation method with a sample frequency F_{S} here chosen at 10 Hz.

The same kind of features may be computed based on breathing or actimetry measurements leading to further stress indices.

For each type of selected measurement and associated timestamped file: heartbeat, breathing or actimetry, at least 3 stress indices, such as average value, standard deviation and BSI may be considered for the machine learning, but the more stress indices the better the prediction, provided that the training set gathers enough experiments.

### Training and Validation

The above computed indices may be categorized and labeled for each window and each subject depending on where it is located in the cycle such as: increasing stress level, labeled e.g. +1, which may correspond to the stressor exercise (813), decreasing stress, label e.g. −1. Which may correspond to the relaxing phase (814) or same stress level, labeled e.g. 0, which may correspond to the rest phase (811) or the neutral phase (815).

The set of records is split into a training set and a validation set. Both sets may be selected based on the behavior of the subjects during the test cycle, notably using the face expression recording, and taking care of an even distribution of criteria such as age, gender and health conditions.

The inventor considered 6 models for machine learning with the training set which where then used to predict stress state evolution using the validation set:
Binary Logistic Regression (BLR)
Support Vector Machine (SVM)
Random Forest (RF)
Linear Discriminant Analysis (LDA)
K-Nearest Neighbors (KNN)
Extreme Gradient Boosting (XGBoost)

Best result is obtained when the algorithm trained with the training set categorized the validation set with the least mistake or the higher success rate.

The inventor found that the XGBoost algorithm brings the best results among the considered algorithms with a success rate ranging from 88% to 94% depending in the validation set.

The above results were obtained on ECG data measured with laboratory style means.

In order to validate the whole method an additional test run was performed using the sensor and the signal processing method as disclosed in the previous sections.

A sample of subjects are subjected to the same testing protocol as shown in FIG. 8 while seating on a chair equipped with a backrest pressure sensor in the backrest and with a seat pressure sensor in the seat. Such arrangement enables to measure physiological parameters and to generate a heartbeat timestamped file, a breath timestamped file and an actimetry timestamped file for the test duration from which features such as disclosed in the feature calculation section may be computed and used through the trained algorithm to get a prediction of the state of stress of the subject.

With 2 sensors, one located in the backrest of the chair and one located in the seat, a blood pressure parameter may also be measured and recorded in a blood pressure timestamped file.

As mentioned before, the BCG measured by the device is more prone to noise than an ECG measured in laboratory like conditions. However, because of the signal processing, the heartbeat timestamped file, the breath timestamped file and the actimetry timestamped file are bringing enough information for enabling features computation and a categorization of a state of stress of an individual using those files alone or in combination.

Based on the heartbeat timestamped file only, the categorization using the trained XGBoost algorithm leads to a 74% success.

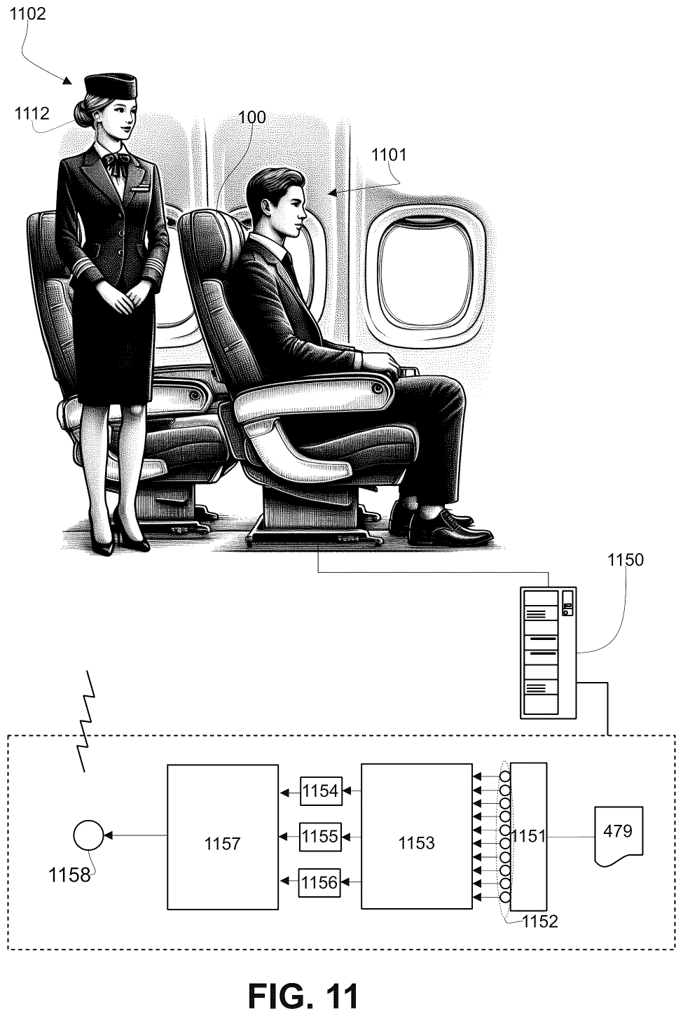

FIG. 11, as an example the method may be implemented in a passenger aircraft wherein passenger seats (100) comprise at least a pressure sensor in the backrest and advantageously both a pressure sensor in the backrest and a pressure sensor in the seating cushion.

A passenger (1101) is seating into the seat (100). Each pressure sensor sends to a computer (1150) a raw signal corresponding to electrical conductivity variation of each elemental gauge of each pressure sensor responsive to the pressure applied by the passenger (1101) on the sensor.

The raw signal is transmitted to the computer along with an identification address comprising at least an information about the seat number in the aircraft cabin. The signal may be transmitted through wired means, for example using the in flight entertainment system of the aircraft, or may be transmitted wireless according to known protocols like WiFi® or BlueTooth®, the signal may be encrypted.

According to an exemplary embodiment the computer comprises an acquisition and digitization board configured to convert each received analog signal in a digital signal, the analog to digital conversion may also be performed before transmitting the signal. The signals of each elemental gauge may also be transferred in a multiplexed signal.

The computer (1150) comprises a non-transient memory in which computer programs are stored for implementing the method of acute stress assessment, starting with signal processing according to the method disclosed in FIG. 4.

This signal processing may separate a pseudo-periodic part and a non-periodic part of the signal. Since the pseudo-periodic part is related to heart beats and breathing of a passenger (1101) seating in the seat, if the intensity of such a pseudo-periodic part of a signal remains under a minimum threshold for more than a given unoccupancy time, for example 10 minutes, the signal processing and associated recordings may be stopped for the corresponding pressure sensors in order to save computing resources, since such a situation may result form a seat being left free, either because there is no passenger assigned to this seat or because the passenger has left the seat for a significant amount of time, and may come back in a different initial state of stress.

As long as a measurement is performed through the disclosed signal processing, at least a heartbeat timestamped file (479) comprising the recording of the IBI of the passenger is generated. Other timestamped files like a breath timestamped file and an actimetry timestamped file may also be generated and stored in a RAM of the computer.

The heartbeat timestamped file for each seat of the aircraft comprising a passenger is scrutinized and a set of indices (1152) is computed from the heartbeat timestamped file (479) by a sliding time assessment window, by a stress indices computing program (1151).

In another embodiment a breath timestamped file as well as an actimetry timestamped file may also be generated and additional features may also be computed from these files according to the same principle.

A stress assessment program (1153) based on an algorithm trained through a machine learning process, assesses a state of stress for each seat, based on the set if indices (1152) thus computed as inputs and categorizes a state of stress into increasing stress (1154), neutral state (1155) and decreasing stress (1156) as outputs.

A monitoring program (1157) follows those outputs for each seat and when the state of stress for a specific seat is found to remain increasing for a set uninterrupted limited time without reverting to neutral or decreasing, the monitoring program (1157), the monitoring program may generate an alarm (1158).

According to various embodiment the alarm may be a visual or an audio signal, in a specific embodiment the monitoring program may broadcast a message through radio means to a member of the crew like a flight attendant (1102) wearing an earflap (1112) for receiving such information. The message may indicate that passenger at seat number xx seems to be subject to a concerning state of acute stress and anxiety. Therefore, the flight attendant (1102) my check the passenger (1101) t see if everything is fine or if the passenger may require some assistance or some advice to cope with stress.

In another embodiment the seats of the pilots of the aircraft, i.e. the captain and the second officer may also be equipped with pressure sensors, enabling a heartbeat timestamped file as well as a breath timestamped file and an actimetry timestamped file to be recorded during the flight and stored in a flight recorder, for instance, in the Cockpit Voice Recorder.

Such an information may be analyzed offline, using a same stress assessment program, as part of an investigation to get a better understanding of human factors that may contribute to a flight incident.

Additionally, those files, measured for the pilots, may also be analyzed in real time during the flight and may be monitored so as trigger an alarm when a sudden increase of stress not followed by a decrease or a neutral state happens, by generating an information that one or the other pilot may be in a state of stress that may affect his decisions.

The above description and preferred embodiments show that the device and the method of the invention make it possible to determine measurable parameters relating to the physiological state of an individual when the latter comes into temporary contact with a surface of a piece of furniture (chair, bed, etc.) equipped with the device of the invention, without it being necessary to install specific sensors coming into direct contact with the skin of the individual.

1. A method for assessing a state of stress of an individual when the individual is contacting a receiving surface of a device comprising a pressure sensor comprising a plurality of elemental gauges, each elemental gauge having a gauge factor of at least 10, the pressure sensor being responsive to a pressure on the receiving surface and delivering a signal to a computer comprising and acquisition and digitization board a non-transient memory and a computer program configured for processing the signal delivered by the pressure sensor, the method comprising steps of:
acquiring and digitizing a signal delivered by an elemental gauge of the pressure sensor the signal comprising a pseudo-periodic part;
in a preprocessing step, extracting the pseudo-periodic part of the signal;
filtering by a bandpass filter with cut-off frequencies of 0.5 Hz and 20 Hz the pseudo-periodic part of the signal obtained in the preprocessing step to obtain a filtered signal;
selecting the filtered signal having a best signal-to-noise ratio among the plurality of elemental gauges;
exploring the filtered signal by a sliding window and detecting, in the sliding window, two patterns corresponding to heartbeats, measuring an Inter-Beat interval of time between the two patterns; and
recording in a heartbeat timestamped file each Inter-Beat Interval (IBI) thus obtained and a corresponding date of measurement.

2. The method of claim 1, wherein the elemental gauge comprises an assembly of electrically conductive nanoparticles in an electrically insulating ligand and two electrically conductive comb shaped electrodes being deposited on the assembly of electrically conductive nanoparticles in a nested interdigitated configuration.

3. The method of claim 2, wherein the gauge factor of the elemental gauge is at least 80.

4. The method of claim 2, wherein the device is in a form of a chair comprising a backrest and a seat and comprising a backrest pressure sensor in the backrest and a seat pressure sensor in the seat.

5. The method of claim 2, wherein the device is in a form of a mattress comprising a sleeping surface and wherein the sleeping surface is the receiving surface.

6. The method of claim 1, comprising the steps of:
from the heartbeat timestamped file computing at least three indices over an assessment time window comprising an average value of the IBI over the assessment time window, a standard deviation of the IBI over the assessment time window and a Baevsky's stress Index from the IBI over the assessment time window; and
categorizing the state of stress of the individual based on the at least 3 indices and a stress assessment program implementing a trained algorithm, among an increasing stress, a decreasing stress and a neutral state;
wherein the assessment time window is comprised between 25 seconds and 250 seconds.

7. The method of claim 6, wherein the trained algorithm is an Extreme Gradient Boosting algorithm.

8. The method of claim 6, wherein a training set for training the trained algorithm is built by:
subjecting a selection of subjects to a stress test comprising at least a neutral phase, an increasing stress phase and a decreasing stress phase; and
continuously recording an IBI of the selection of subjects during their performance of the stress test;
wherein a duration of the stress test is comprised between 10 minutes and 30 minutes and the duration of each phase is at least 4 minutes.

9. The method of claim 8, wherein a continuous recording of an IBI of a subject is split in windows of a duration comprised between 25 seconds and 250 seconds, a beginning of each window being separated from a beginning of a previous window by 5 seconds, the at least 3 indices being computed for each window.

10. The method of claim 8, wherein the increasing stress phase is obtained by subjecting the selection of subjects to a stressor exercise.

11. The method of claim 10, wherein the stressor exercise comprises a modified Stroop test wherein the selection of subjects is given a limited time to perform each Stroop test exercise.

12. The method of claim 8, wherein the device is in a form of an aircraft seat and the individual is seating on the aircraft seat, the method comprising a step of:
If the state of stress of the individual is categorized as an increasing state of stress for more than an uninterrupted limited time set, triggering an alarm.

13. The method of claim 12, wherein a recording of the heartbeat timestamped file is stopped if an intensity of the pseudo-periodic part of the signal of the plurality of elemental gauges remains under a minimum threshold for more than a given unoccupancy time.

14. The method of claim 8, wherein the device is in a form of an aircraft seat and the individual is a pilot seating on the aircraft seat, the heartbeat timestamped file being recorded in a flight data recorder.
